(19) United States
(12) Patent Application Publication
(10) Pub. No.: US 2024/0117444 A1
(43) Pub. Date: Apr. 11, 2024

(54) BIOMARKERS FOR THE DIAGNOSIS OF BREAST CANCER

(71) Applicant: MAMMOGEN, INC., Newport Beach, CA (US)

(72) Inventors: Patrick LILLEY, Newport Beach, CA (US); Martin J. KEISER, III, Fairfield, CT (US)

(73) Assignee: MAMMOGEN, INC., Newport Beach, CA (US)

(21) Appl. No.: 18/546,333

(22) PCT Filed: Feb. 11, 2022
(86) PCT No.: PCT/US22/16195

§ 371 Date: Aug. 14, 2023

Related U.S. Application Data
(60) Provisional application No. 63/247,980, filed on Sep. 24, 2021.
(60) Provisional application No. 63/149,181, filed on Feb. 12, 2021.

(51) Int. Cl. C12Q 1/6886

(52) U.S. Cl. CPC C12Q 1/6886; C12Q 2600/112; C12Q 2600/158

(57) ABSTRACT

Embodiments of the invention include a system and method of using biomarkers in the diagnosis of disease, for example, breast cancer. A subject can be screened for breast cancer based on altered expression of one or more mRNAs in blood, plasma or salvia. Embodiments include 26 specific mRNAs for use as biomarkers to screen or distinguish healthy individuals from individuals affected with breast cancer. Levels of more than one of the mRNAs can be used to create a biomarker fingerprint for early screening of a breast cancer. Embodiments also include a kit for screening healthy subjects from subjects affected with breast cancer.

### FIELD OF THE INVENTION

The invention relates to the diagnosis of disease using biomarkers, and more specifically, to a system and method of diagnosing breast cancer based on altered expression of one or more specific mRNAs.

### BACKGROUND

Breast cancer is cancer that develops from breast tissue. Worldwide, breast cancer is the leading type of cancer in women, accounting for 25% of all cases. In 2018 it resulted in 2 million new cases and 627,000 deaths. Risk factors for developing breast cancer include being female, obesity, a lack of physical exercise, alcoholism, hormone replacement therapy during menopause, ionizing radiation, an early age at first menstruation, having children late in life or not at all, older age, having a prior history of breast cancer, and a family history of breast cancer. About 5-10% of cases are the result of a genetic predisposition inherited from a person's parents, including BRCA1 and BRCA2 among others.

Breast cancer most commonly presents as a lump that feels different from the rest of the breast tissue. More than 80% of cases are discovered when a person detects such a lump with the fingertips. The earliest breast cancers, however, are detected by a mammogram. Lumps found in lymph nodes located in the armpits may also indicate breast cancer.

Breast cancer can begin in different parts of the breast. A breast is made up of three main parts: lobules, ducts, and connective tissue. The lobules are the glands that produce milk. The ducts are tubes that carry milk to the nipple. The connective tissue (which consists of fibrous and fatty tissue) surrounds and holds everything together. Most breast cancers begin in the ducts or lobules. Breast cancer most commonly presents as a lump that feels different from the rest of the breast tissue. Breast cancer can spread outside the breast through blood vessels and lymph vessels. When breast cancer spreads to other parts of the body, it is said to have metastasized.

The diagnosis of breast cancer can be confirmed by taking a biopsy of the suspect tissue. Once the diagnosis is made, further tests can determine if the cancer has spread beyond the breast and which treatments are most likely to be effective. Outcomes for breast cancer vary depending on the cancer type, the extent of disease, and the person's age. The five-year survival rates in England and the United States are between 80 and 90%. In developing countries, five-year survival rates are lower.

In those who have been diagnosed with cancer, a number of treatments can be used, including surgery, radiation therapy, chemotherapy, hormonal therapy, and targeted therapy. Types of surgery vary from breast-conserving surgery to mastectomy. Breast reconstruction may take place at the time of surgery or at a future date. In patients wherein the cancer has spread to other parts of the body, treatments are mostly aimed at improving quality of life and comfort.

Early detection of breast cancer is essential to effective treatment. According to the American Cancer Society, when breast cancer is detected early, and is in the localized stage, the five-year relative survival rate is 99%. Conventional methods of early detection include monthly breast self-exams, regular clinical breast exams and mammograms. However, methods of early detection have limitations.

Currently, breast cancers are generally detected by a mammogram. A number of national bodies recommend breast cancer screening. For the average woman, the U.S. Preventive Services Task Force and American College of Physicians recommends mammography every two years in women between the ages of 50 and 74, the Council of Europe recommends mammography between 50 and 69 with most programs using a 2-year frequency, while the European Commission recommends mammography from 45 to 75 every 2 to 3 years, and in Canada screening is recommended between the ages of 50 and 74 at a frequency of 2 to 3 years. Following detection, the diagnosis of breast cancer is confirmed by taking a biopsy of the tissue where breast cancer is suspected. Once the diagnosis is made, further tests are conducted to determine if the cancer has spread beyond the breast and which therapeutic treatments are most likely to be effective.

The use of mammography as a screening tool for the detection of early breast cancer in otherwise healthy women without symptoms is controversial. Adverse effects of errors in diagnosis, over-treatment, and radiation exposure. The balance of benefits versus harms of breast cancer screening is controversial. A 2013 Cochrane review found that it was unclear if mammographic screening does more harm than good, in that a large proportion of women had false positive test results.

Biomarkers are a non-invasive and cost-effective means to aid in clinical management of cancer patients, particularly in areas of disease detection, prognosis, monitoring and therapeutic stratification. For a serological biomarker to be useful for early detection, its presence in serum must be relatively low in healthy individuals and those with benign disease. The biomarker must be produced by the tumor or its microenvironment and enter circulation, giving rise to increased serum levels. Mechanisms that facilitate entry to circulation include secretion or shedding, angiogenesis, invasion, and destruction of tissue architecture. The biomarker should preferably be tissue specific, such that a change in serum level can be directly attributed to disease (e.g., cancer) of that tissue.

For example, serum PSA is commonly used for prostate cancer screening in men over 50, but its usage remains controversial due to serum elevation in benign disease as well as prostate cancer. Nevertheless, PSA represents one of the most useful serological markers currently available. PSA is strongly expressed in only the prostate tissue of healthy men, with low levels in serum established by normal diffusion through various anatomical barriers. These anatomical barriers are disrupted upon development of prostate cancer, allowing increased amounts of PSA to enter circulation.

Other common serological biomarkers include carcinoembryonic antigen (CEA) and carbohydrate antigen 19.9 (CA19.9) for gastrointestinal cancer, CEA, CYFRA 21-1 (cytokeratin 19 fragment), neuron-specific enolase (NSE), tissue polypeptide antigen (TPA), progastrin-releasing peptide (pro-GRP), and SCC antigen for lung cancer, CA 125 for ovarian cancer and prostate-specific antigen (PSA, also known as KLK3) in prostate cancer. However, these biomarkers have limitations and generally lack the appropriate sensitivity and specificity to be suitable for early cancer detection. Further, there are no biomarkers available for detecting breast cancer.

Because of the limitations of accurately detecting breast cancer, it is difficult to study disease progression and develop drugs/therapies. Moreover, it is difficult for drug developers to properly recruit patients for their clinical trials. Further, patients are generally averse to undergoing a breast biopsy because of its invasiveness and risks. Accordingly, there is a need for accurate and affordable noninvasive breast cancer tests.

There is a need for improved diagnostic assays and methods of detecting cancer, specifically breast cancer. Conventional diagnostic assays often rely on a single biomarker and are unreliable in detecting the presence of cancer or tumor progression. Thus, there is a need for the identification of alternative molecular markers that overcome these limitations.

### SUMMARY OF THE INVENTION

The following summary is provided to facilitate an understanding of some of the innovative features unique to the disclosed embodiment and is not intended to be a full description. A full appreciation of the various aspects of the embodiments disclosed herein can be gained by taking into consideration the entire specification, claims and abstract as a whole.

Embodiments include a system and method of detection and diagnosis of breast cancer and its progression.

Embodiments include a set of peripheral diagnostic biomarkers for detecting breast cancer. Embodiments include mRNA biomarkers to detect breast cancer. Embodiments also include mRNA biomarkers to detect breast cancer early in its progression.

Additional embodiments include mRNA biomarkers to distinguish between the types of breast cancer. Embodiments include mRNA biomarkers to distinguish between different genetic forms of breast cancer. The breast cancer can be, for example, ductal carcinoma in situ, invasive lobular carcinoma in situ, tubular/cribriform carcinoma, mucinous (colloid) carcinoma, medullary carcinoma, papillary carcinoma and metaplastic carcinoma.

Embodiments include mRNA biomarkers to distinguish between a breast cancer and a healthy breast. Embodiments also include the use of nucleic acids, proteins and/or peptides (e.g., as identified in Table 1) to distinguish between a breast cancer and a healthy breast.

Embodiments include mRNA biomarkers to monitor the progress of breast cancer in a patient. Embodiments include mRNA biomarkers to provide guidance in choosing among one or more therapies and/or drugs to treat a patient with breast cancer.

Embodiments include a system and method of determining a preferred treatment for a patient suffering from breast cancer. Embodiments also include a system and method of determining a patient's likelihood of responding favorably to surgical procedures such as mastectomy.

Embodiments include a method that uses one or more algorithms to diagnose a breast cancer based on levels of one or more mRNA biomarkers.

Embodiments include a method that uses one or more algorithms to diagnose a breast cancer based on levels of one or more mRNA biomarkers.

Embodiments also include a method that uses one or more algorithms to diagnose a breast cancer based on levels of one or more protein biomarkers.

Embodiments include methods that use one or more biomarkers to distinguish between stage 0, stage 1, stage 2, stage 3, stage 4 and stage 5 of breast cancer.

The methods and assays disclosed herein are directed to the examination of the amount of one or more biomarkers in a biological sample, wherein the determination of that amount of one or more such biomarkers is predictive or indicative of the presence of breast cancer. The disclosed methods and assays provide for convenient, efficient, and potentially cost-effective means to obtain data and information useful in assessing appropriate or effective therapies for treating patients.

Methods for detecting any biomarkers desired to be assessed include protocols that examine the presence and/or expression of a desired nucleic acid. Tissue or cell samples from mammals can be conveniently assayed for, e.g., genetic-marker mRNAs or DNAs using Northern, dot-blot, or polymerase chain reaction (PCR) analysis, array hybridization, RNase protection assay, or using DNA SNP chip microarrays, which are commercially available, including DNA microarray snapshots. For example, real-time PCR (RT-PCR) assays such as quantitative PCR assays are well known in the art.

More specifically, embodiments include methods of detecting breast cancer based on specific genes/mRNAs that have altered expression levels. The Applicant has identified 26 specific mRNAs that can be used as biomarkers to distinguish healthy individuals from individuals affected with breast cancer. The use of biomarkers is non-invasive and potentially more sensitive than conventional methods. Types of breast cancer include ductal carcinoma in situ, invasive lobular carcinoma in situ, tubular/cribriform carcinoma, mucinous (colloid) carcinoma, medullary carcinoma, papillary carcinoma and metaplastic carcinoma. Embodiments also include methods of prognosis, patient monitoring and distinguishing among different types of breast cancer. Based on the prognosis, an appropriate treatment plan can be devised.

Embodiments also include the use of one or more of the following genes (or mRNAs) as biomarkers: SLC8A1, RP11-452L6.1, HINT3, PRORSD1P, PRDX6, GABRB2, EPS8, SH3RF1, GB_ACC AA814006, Hypothetical protein LOC284058, ZNF160, SLC25A45, DST, MTAP, GB_Acc AK024901, TFB1M, SPRED1, CAV1, MUC1, NAP1L3, MDH1B, TMEM125, PDE9A, TSHZ2, LAMB4 and PTGS2.

Embodiments include a method of detecting cancer or determining a prognosis of a subject with cancer (such as breast cancer), that includes steps of a) measuring the expression level of at least one mRNA in a test sample from plasma of the subject; b) comparing the expression level of the mRNA in the test sample to a level in a base sample; and c) detecting or determining the prognosis of cancer based on altered expression the mRNA in the test sample. The method can distinguish between different stages (corresponding to stage 0-5) of breast cancer. The method can also include a step of treating the cancer based on the detection/prognosis. Common treatments include surgery, radiation, chemotherapy, hormone therapy, targeted drug therapy and/or immunotherapy.

Embodiments also include a method of detecting cancer or determining a prognosis of a subject with cancer (such as breast cancer), that includes steps of a) measuring the expression level of at least one mRNA in a test sample from plasma of the subject; b) comparing the expression level of the mRNA in the test sample to a level in a base sample; and c) detecting or determining the prognosis of cancer based on altered expression the mRNA in the test sample. The method can include a step of treating the cancer based on the detection/prognosis. The method can be used with other bodily fluids including saliva.

Embodiments also include a method of detecting cancer or determining a prognosis of a test subject with cancer (such as breast cancer), that includes steps of: a) measuring expression levels of two or more mRNAs in plasma samples from subjects with cancer; b) measuring expression levels of the same mRNAs in plasma samples from healthy subjects; c) comparing the expression levels of the mRNAs in the plasma samples from the subjects with cancer to the levels in the plasma samples from the healthy subjects; d) identifying mRNAs that have altered levels of expression in the plasma samples from the subjects with cancer; e) creating a biomarker fingerprint from the mRNAs with altered levels of expression; and f) diagnosing or determining the prognosis of cancer in the test subject by comparing of levels of mRNAs from plasma of the test subject to those in the biomarker fingerprint. The method can also include a step of treating the cancer based on the detection/prognosis.

Embodiments also include a method of diagnosing cancer or determining a prognosis of a test subject with cancer (such as breast cancer), that includes steps of: a) measuring expression levels of two or more mRNAs in saliva obtained from blood from subjects with cancer; b) measuring expression levels of the two or more mRNAs in saliva obtained from blood from samples from healthy subjects; c) comparing the expression levels of the two or more mRNAs in the saliva obtained from the subjects with cancer to the levels in the plasma samples from the healthy subjects; d) identifying mRNAs that have altered levels of expression in the saliva obtained from blood from samples from the subjects with cancer; e) creating a biomarker fingerprint from the mRNAs with altered levels of expression; and f) diagnosing or determining the prognosis of cancer in the test subject by comparing of levels of mRNAs from plasma of the test subject to those in the biomarker fingerprint. The method can include a step of treating the cancer based on the detection/prognosis.

Embodiments also include a diagnostic kit for diagnosing breast cancer or detecting the presence of a tumor. The kit can detect the presence of breast cancer tumor cells in plasma or saliva from a patient. The kit can include a plurality of nucleic acid molecules, each nucleic acid molecule encoding a mRNA sequence. The nucleic acid molecules identify variations in expression levels of one or more mRNAs in a plasma or saliva sample from a test subject. The expression levels of one or more mRNAs can represent a nucleic acid expression fingerprint that is indicative for the presence of a tumor or breast cancer.

Embodiments also include a method for identifying one or more mammalian target cells exhibiting breast cancer that includes steps of: a) collecting plasma from a test subject; b) hybridizing at least one nucleic acid molecule biomarker encoding a mRNA sequence to a portion of the plasma; c) quantifying the mRNA expression; d) determining the expression levels of a plurality of nucleic acid molecules, each nucleic acid molecule encoding a mRNA sequence; e) determining the expression levels of the plurality of nucleic acid molecules in one or more control cells; and f) identifying from the plurality of nucleic acid molecules one or more nucleic acid molecules that are differentially expressed in the target and control cells by comparing the respective expression levels obtained in steps (d) and (e). The method can include a step of treating the cancer based on the detection/prognosis. The differentially expressed nucleic acid molecules together can represent a nucleic acid expression biomarker fingerprint that is indicative of the presence of breast cancer.

### Definitions

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can be in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

As applicable, the terms “about” or “generally”, as used herein in the specification and appended claims, and unless otherwise indicated, means a margin of +1-20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

The term “algorithm” refers to a specific set of instructions or a definite list of well-defined instructions for carrying out a procedure, typically proceeding through a well-defined series of successive states, and eventually terminating in an end-state.

The term “biomarker” refers generally to a DNA, RNA, protein, carbohydrate, or glycolipid-based molecular marker, the expression or presence of which in a subject's sample can be detected by standard methods (or methods disclosed herein) and is predictive or prognostic of the effective responsiveness or sensitivity of a mammalians subject with cancer. Biomarkers may be present in a test sample but absent in a control sample, absent in a test sample but present in a control sample, or the amount or of biomarker can differ between a test sample and a control sample. For example, genetic biomarkers assessed (e.g., specific mutations and/or SNPs) can be present in such a sample, but not in a control sample, or certain biomarkers are seropositive in the sample, but seronegative in a control sample. Also, optionally, expression of such a biomarker may be determined to be higher than that observed for a control sample. The terms “marker” and “biomarker” are used herein interchangeably.

As used herein, “additional biomedical information” refers to one or more evaluations of an individual, other than using any of the biomarkers described herein, that are associated with breast cancer risk. “Additional biomedical information” includes any of the following: physical descriptors of an individual, physical descriptors of a pulmonary nodule observed by CT imaging, the height and/or weight of an individual, the gender of an individual, the ethnicity of an individual, smoking history, occupational history, exposure to known carcinogens (e.g., exposure to any of asbestos, radon gas, chemicals, smoke from fires, and air pollution, which can include emissions from stationary or mobile sources such as industrial/factory or auto/marine/aircraft emissions), exposure to second-hand smoke, family history of breast cancer (or other cancer), the presence of pulmonary nodules, size of nodules, location of nodules, morphology of nodules (e.g., as observed through CT imaging, ground glass opacity (GGO), solid, non-solid), edge characteristics of the nodule (e.g., smooth, lobulated, sharp and smooth, spiculated, infiltrating), and the like. Additional biomedical information can be obtained from an individual using routine techniques known in the art, such as from the individual themselves by use of a routine patient questionnaire or health history questionnaire, etc., or from a medical practitioner, etc. Alternately, additional biomedical information can be obtained from routine imaging techniques, including CT imaging (e.g., low-dose CT imaging) and X-ray. Testing of biomarker levels in combination with an evaluation of any additional biomedical information may, for example, improve sensitivity, specificity, and/or AUC for detecting breast cancer (or other breast cancer-related uses) as compared to biomarker testing alone or evaluating any particular item of additional biomedical information alone (e.g., CT imaging alone).

The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g., breast cancer samples and normal or control samples). ROC curves are useful for plotting the performance of a particular feature (e.g., any of the biomarkers described herein and/or any item of additional biomedical information) in distinguishing between two populations (e.g., cases having breast cancer and controls without breast cancer). Typically, the feature data across the entire population (e.g., the cases and controls) are sorted in ascending order based on the value of a single feature. Then, for each value for that feature, the true positive and false positive rates for the data are calculated. The true positive rate is determined by counting the number of cases above the value for that feature and then dividing by the total number of cases. The false positive rate is determined by counting the number of controls above the value for that feature and then dividing by the total number of controls. Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to provide a single sum value, and this single sum value can be plotted in a ROC curve. Additionally, any combination of multiple features, in which the combination derives a single output value, can be plotted in a ROC curve. These combinations of features may comprise a test. The ROC curve is the plot of the true positive rate (sensitivity) of a test against the false positive rate (1-specificity) of the test.

As used herein, “detecting” or “determining” with respect to a biomarker value includes the use of both the instrument required to observe and record a signal corresponding to a biomarker value and the material/s required to generate that signal. In various embodiments, the biomarker value is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

The term “prognosis” refers to the forecast or likely outcome of a disease. As used herein, it refers to the probable outcome of breast cancer, including whether the disease will respond to treatment or mitigation efforts and/or the likelihood that the disease will progress.

The term “fingerprint,” “disease fingerprint,” or “biomarker signature” refers to a plurality or pattern of biomarkers that have elevated or reduced levels in a subject with disease. A fingerprint can be generated by comparing subjects with the disease to healthy subjects and used for screening/diagnosis of the disease.

The term “miRNA” or “micro RNA,” “miRNA biomarkers,” or “MicroRNAs” refers to small endogenous RNA molecules that can be used as serum diagnostic biomarkers for diseases including cancers.

The term “messenger RNA” or “mRNA” refers to a single-stranded molecule of RNA that corresponds to the genetic sequence of a gene and is read by a ribosome in the process of synthesizing a protein. As used herein, mRNA can also include “miRNA” and small interfering RNAs (siRNAs).

An mRNA that is “unregulated” generally refers to an increase in the level of express of the mRNA in response to a given treatment or condition. An mRNA that is “downregulated” generally refers to a “decrease” in the level of expression of the mRNA in response to a given treatment or condition. In some situations, the mRNA level can remain unchanged upon a given treatment or condition. An mRNA from a patient sample can be “unregulated,” (i.e., the level of mRNA can be increased). Alternatively, an mRNA can be “downregulated” (i.e., the level of mRNA level can be decreased).

An mRNA that is “unregulated” generally refers to an increase in the level of express of the mRNA in response to a given treatment or condition. An mRNA that is “downregulated” generally refers to a “decrease” in the level of expression of the mRNA in response to a given treatment or condition. In some situations, the mRNA level can remain unchanged upon a given treatment or condition. An mRNA from a patient sample can be “unregulated,” i.e., the level of mRNA can be increased, for example, by about 5%, about 10%, about 20%, about 30%, about 40%, about 50%, about 60%, about 70%, about 90%, about 100%, about 200%, about 300%, about 500%, about 1,000%, about 5,000% or more of the comparative control mRNA level or a reference level. Alternatively, an mRNA can be “downregulated,” i.e., the level of mRNA level can be decreased, for example, by about 99%, about 95%, about 90%, about 80%, about 70%, about 60%, about 50%, about 40%, about 30%, about 20%, about 10%, about 5%, about 2%, about 1% or less of the comparative control mRNA level or a reference level.

Similarly, the level of a polypeptide, protein, or peptide from a patient sample can be increased as compared to a control or a reference level. This increase can be about 5%, about 10%, about 20%, about 30%, about 40%, about 50%, about 60%, about 70%, about 90%, about 100%, about 200%, about 300%, about 500%, about 1,000%, about 5,000% or more of the comparative control protein level or a reference level. Alternatively, the level of a protein biomarker can be decreased. This decrease can be, for example, present at a level of about 99%, about 95%, about 90%, about 80%, about 70%, about 60%, about 50%, about 40%, about 30%, about 20%, about 10%, about 5%, about 2%, about 1% or less of the comparative control protein level or a reference level.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Methods for obtaining (e.g., producing, isolating, purifying, synthesizing, and recombinantly manufacturing) polypeptides are well known to one of ordinary skill in the art. The level of a polypeptide, protein, or peptide from a patient sample can be increased as compared to a control or a reference level. Alternatively, the level of a protein biomarker can be decreased.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “medicament” refers to an active drug to treat cancer, such as breast cancer, or the signs or symptoms or side effects of cancer.

The term “plasma” or “blood plasma” refers to the liquid portion of the blood that carries cells and proteins throughout the body. Plasma can be separated from the blood by spinning a tube of fresh blood containing an anticoagulant in a centrifuge until the blood cells fall to the bottom of the tube.

The term “PCR” or “polymerase chain reaction” refers to a common method used to make many copies of a specific DNA segment. Variations of the technique can be used to determine the presence and amount of one or more mRNAs in a sample. For example, a hydrolysis probe-based stem-loop quantitative reverse-transcription PCR (RT-qPCR) assay can be conducted to confirm and/or quantify the concentrations of selected mRNAs in serum samples from patients and controls.

The term “sample” refers to a biological sample obtained from an individual, body fluid, body tissue, cell line, tissue culture, or other source. Body fluids are, for example, lymph, sera, whole fresh blood, peripheral blood mononuclear cells, frozen whole blood, plasma (including fresh or frozen), urine, saliva, semen, synovial fluid and spinal fluid. Samples also include synovial tissue, skin, hair follicle, and bone marrow. Methods for obtaining tissue biopsies and body fluids from mammals are well known in the art.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

The term “nucleic acid probe” or “oligonucleotide probe” refers to a nucleic acid capable of binding to a target nucleic acid of complementary sequence, such as the mRNA biomarkers provided herein, through one or more types of chemical bonds, usually through complementary base pairing, usually through hydrogen bond formation. As used herein, a probe may include natural (e.g., A, G, C, or T) or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences lacking complete complementarity with the probe sequence depending upon the stringency of the hybridization conditions. The probes are preferably directly labeled with isotopes, for example, chromophores, lumiphores, chromogens, or indirectly labeled with biotin to which a streptavidin complex may later bind. By assaying for the presence or absence of the probe, one can detect the presence or absence of a target mRNA biomarker of interest.

The term “probe id.” “probe set identifier,” or “Affymetrix probe set ID” refers to the identifier that refers to a set of probe pairs selected to represent expressed sequences on an array. (_at =all the probes hit one known transcript; _a=all probes in the set hit alternate transcripts from the same gene; _s=all probes in the set hit transcripts from different genes; _x=some probes hit transcripts from different genes).

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### DETAILED DESCRIPTION

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the structure particularly pointed out in the written description and claims hereof.

Conventional methods of diagnosing breast cancer are not necessarily reliable and involve invasive procedures such as tumor biopsy. Currently, breast cancers are generally detected by a mammogram, which upon the diagnosis of breast cancer is confirmed by taking a biopsy of the tissue where breast cancer is suspected.

Recent studies have demonstrated the presence of mRNAs in saliva and circulating blood and their potential for use as biomarkers in the diagnosis of various diseases. Specifically, efforts have proposed their use as biomarkers for early detection of cancers such as breast cancer.

The Applicant has recognized mRNA expression profiling in subjects with breast cancer. Specific mRNAs are aberrantly expressed in malignant tissues as compared to nonmalignant breast tissue. Moreover, mRNA expression can provide insights into cellular processes involved in the malignant transformation and progression. Thus, mRNA expression levels can also be used for breast cancer prognosis. Specifically, the technology provides diagnostic methods for predicting and/or prognosticating the effectiveness of treatment.

The present invention is based on the finding that breast cancer can be reliably identified based on particular mRNA expression profiles with high sensitivity and specificity. The expression of biomarkers typically includes both up- and down-regulated levels of mRNAs. An analysis of mRNA expression biomarkers allows for creation of a “fingerprint” by analyzing mRNA expression patterns in diseased and healthy subjects. Thereafter, individual mRNA expression levels can be used for the detection of breast cancer at early stages of the disease. The biomarkers can also be used to distinguish different subtypes of breast cancer from one another and monitor the progress of a breast cancer. Additional embodiments include mRNA biomarkers to distinguish between different stages (corresponding to stage 0-5) of breast cancer. Further embodiments include mRNA biomarkers to distinguish between the types of breast cancer. Embodiments include mRNA biomarkers to distinguish between different genetic forms of breast cancer. In another embodiment, the proteins transcribed from identified mRNAs are used as biomarkers.

Early diagnosis of breast cancer allows intervention and/or treatment to avoid further damage, including the metastases of the breast cancer cells into other tissues and organs. For example, the biomarkers described herein can be measured and analyzed to determine whether a patient has a healthy breast without cancerous tissue or has breast cancer, including diagnosing in the early stages of breast cancer. Conventional methods of diagnosing breast cancer rely on touching the breast to identify lumps or the use of mammograms to identify cancerous lumps. Because the early stages of breast cancer is often asymptomatic, these methods may be ineffective to identify patients suffering from breast cancer at a stage when treatment would have the greatest effect. Early, accurate and reliable detection of breast cancer can be utilized by researchers and drug developers to recruit patients for clinical trials, support their drug through development, and support the drug post-approval.

The disclosed biomarkers can also be used to monitor the progression of breast cancer. For example, the biomarkers described herein can be measured and analyzed to identify the stage of breast cancer. Breast cancer can be one of five stages: Stage 0, Stage 1, Stage 2, Stage 3 and Stage 4. When possible, efforts can be made to slow (or reverse) the progress of the disease. Conventional methods lack the reliability and sensitivity to distinguish between these conditions and monitor progression.

Embodiments include a set of diagnostic markers or a molecular fingerprint, for quick and reliable identification and/or treatment of cells exhibiting or having a predisposition to develop different subtypes of breast cancer. Embodiments further include methods of diagnosing cancer based on specific mRNAs that have altered expression levels. While individual mRNAs can be monitored, the invention includes 26 mRNAs of particular value as biomarkers to screen or distinguish healthy individuals from individuals affected with disease. The mRNAs of particular interest (detailed in Table 1) include: SLC8A1, RP11-452L6.1, HINT3, PRORSD1P, PRDX6, GABRB2, EPS8, SH3RF1, GB_ACC AA814006, Hypothetical protein LOC284058, ZNF160, SLC25A45, DST, MTAP, GB_Acc AK024901, TFB1M, SPRED1, CAV1, MUC1, NAP1L3, MDH1B, TMEM125, PDE9A, TSHZ2, LAMB4 and PTGS2.

Embodiments include a method of identifying an initial set of peripheral diagnostic biomarkers for detecting breast cancer and validating these biomarkers on additional datasets, including saliva and peripheral blood. The method can be implemented by the steps of:
a) Producing binary classifiers to distinguish between patients with a confirmed positive breast cancer biopsy and patients with a confirmed negative breast cancer biopsy;
b) Identifying the first minimal set of biomarkers that maximizes diagnostic accuracy;
c) Setting aside the first minimal set and repeat the analysis with the remaining biomarkers, to determine the next minimal set; and
d) Repeating the above step until observing a significant decline in diagnostic accuracy.

The methods and materials can be used for assessing subjects (e.g., human patients) for cancer such as breast cancer. For example, embodiments include materials and methods for using identifiable markers to assist clinicians in assessing breast cancer disease activity, assessing the likelihood of response and outcomes of therapy, and predicting long-term disease outcomes. Further, subjects with breast cancer can be diagnosed based on the presence of certain diagnostic indicators in plasma or saliva from the subject. Thus, the technology allows for the diagnosis of breast cancer based on one or more combinations of markers.

The severity of breast cancer can be described as falling within one of five stages. Stage 0 is a pre-cancerous or marker condition, which is commonly associated with either ductal carcinoma in situ (DCIS) or lobular carcinoma in situ (LCIS). Stages 1-3 the tumors and/or cancer cells are found within the breast or regional lymph nodes. When an individual is diagnosed with Stage 4 breast cancer, this means that the breast cancer has become a ‘metastatic’ cancer wherein the cancer cells have spread to other tissues and/or organs of the patient. Patients diagnosed with Stage 4 breast cancer have a less favorable prognosis than those diagnosed in Stages 0-3, since the cancer cells have spread beyond the breast and lymph nodes located proximal to the breast where the cancer is located.

Multiple mRNA biomarkers can be used from a single serum or saliva sample taken from a subject. According to some embodiments, multiple biomarkers are assessed and measured from different samples taken from the patient. According to some embodiments, the subject technology is used for a kit for predicting, diagnosing or monitoring responsiveness of a cancer treatment or therapy, wherein the kit is calibrated to measure marker levels in a sample from the patient.

According to some embodiments, the amount of biomarkers can be determined by using, for example, a reagent that specifically binds with the biomarker protein or a fragment thereof, (e.g., an antibody, a fragment of an antibody, or an antibody derivative). The level of expression can be determined using a method common in the art such as proteomics, flow cytometry, immunocytochemistry, immunohistochemistry, enzyme-linked immunosorbent assay, multi-channel enzyme linked immunosorbent assay, and variations thereof. The expression level of a biomarker in the biological sample can also be determined by detecting the level of expression of a transcribed biomarker polynucleotide or fragment thereof encoded by a biomarker gene, which may be cDNA, mRNA or heterogeneous nuclear RNA (hnRNA). The step of detecting can include amplifying the transcribed biomarker polynucleotide and can use the method of quantitative reverse transcriptase polymerase chain reaction. The expression level of a biomarker can be assessed by detecting the presence of the transcribed biomarker polynucleotide or a fragment thereof in a sample with a probe which anneals with the transcribed biomarker polynucleotide or fragment thereof under stringent hybridization conditions.

Also provided herein are compositions and kits for practicing the methods. For example, in some embodiments, reagents (e.g., primers, probes) specific for one or more markers are provided alone or in sets (e.g., sets of primers pairs for amplifying a plurality of markers). Additional reagents for conducting a detection assay may also be provided (e.g., enzymes, buffers, positive and negative controls for conducting QuARTS, PCR, sequencing, bisulfite, or other assays). In some embodiments, the kits containing one or more reagent necessary, sufficient, or useful for conducting a method are provided. Also provided are reactions mixtures containing the reagents. Further provided are master mix reagent sets containing a plurality of reagents that may be added to each other and/or to a test sample to complete a reaction mixture.

In some embodiments, the technology described herein is associated with a programmable machine designed to perform a sequence of arithmetic or logical operations as provided by the methods described herein. For example, some embodiments of the technology are associated with (e.g., implemented in) computer software and/or computer hardware. In one aspect, the technology relates to a computer comprising a form of memory, an element for performing arithmetic and logical operations, and a processing element (e.g., a microprocessor) for executing a series of instructions (e.g., a method as provided herein) to read, manipulate, and store data. Therefore, certain embodiments employ processes involving data stored in or transferred through one or more computer systems or other processing systems. Embodiments disclosed herein also relate to apparatus for performing these operations. This apparatus may be specially constructed for the required purposes, or it can be a general-purpose computer (or a group of computers) selectively activated or reconfigured by a computer program and/or data structure stored in the computer. In some embodiments, a group of processors performs some or all of the recited analytical operations collaboratively (e.g., via a network or cloud computing) and/or in parallel.

In some embodiments, a microprocessor is part of a system for determining the presence of one or more mRNA or miRNAs (labeled herein as hsa-miR or has-miRs) associated with a cancer; generating standard curves; determining a specificity and/or sensitivity of an assay or marker; calculating an ROC curve; sequence analysis; all as described herein or is known in the art.

In some embodiments, a microprocessor is part of a system for determining the amount, such as concentration, of one or more mRNAs associated with a cancer; generating standard curves; determining a specificity and/or sensitivity of an assay or marker; calculating an ROC curve; sequence analysis; all as described herein or is known in the art. The amount of one or more mRNAs can be determined by abundance, measured per mole or millimole. The amount of mRNAs can be determined by fluorescence, other measurement using an optical signal or other measurement known to one of skill to measure levels of mRNAs.

In some embodiments, a microprocessor or computer uses an algorithm to measure the amount of an mRNA or multiple mRNAs. The algorithm can include a mathematical interaction between a marker measurement or a mathematical transform of a marker measurement. The mathematical interaction and/or mathematical transform can be presented in a linear, nonlinear, discontinuous or discrete manner.

In some embodiments, a software or hardware component receives the results of multiple assays and determines a single value result to report to a user that indicates a cancer risk based on the results of the multiple assays. Related embodiments calculate a risk factor based on a mathematical combination (e.g., a weighted combination, a linear combination) of the results from multiple assays as disclosed herein.

Some embodiments include a storage medium and memory components. Memory components (e.g., volatile and/or nonvolatile memory) find use in storing instructions (e.g., an embodiment of a process as provided herein) and/or data (e.g., a work piece such as methylation measurements, sequences, and statistical descriptions associated therewith). Some embodiments relate to systems also comprising one or more of a CPU, a graphics card, and a user interface (e.g., comprising an output device such as display and an input device such as a keyboard).

Programmable machines associated with the technology comprise conventional extant technologies and technologies in development or yet to be developed (e.g., a quantum computer, a chemical computer, a DNA computer, an optical computer, a spintronics based computer, etc.).

In some embodiments, the technology comprises a wired (e.g., metallic cable, fiber optic) or wireless transmission medium for transmitting data. For example, some embodiments relate to data transmission over a network (e.g., a local area network (LAN), a wide area network (WAN), an ad-hoc network, the internet, etc.). In some embodiments, programmable machines are present on such a network as peers and in some embodiments the programmable machines have a client/server relationship.

In some embodiments, data are stored on a computer-readable storage medium such as a hard disk, flash memory, memory stick, optical media, a floppy disk, etc.

In some embodiments, the technology provided herein is associated with a plurality of programmable devices that operate in concert to perform a method as described herein. For example, in some embodiments, a plurality of computers (e.g., connected by a network) may work in parallel to collect and process data, e.g., in an implementation of cluster computing or grid computing or some other distributed computer architecture that relies on complete computers (with onboard CPUs, storage, power supplies, network interfaces, etc.) connected to a network (private, public, or the internet) by a conventional network interface, such as Ethernet, fiber optic, or by a wireless network technology.

For example, some embodiments provide a computer that includes a computer-readable medium. The embodiment includes a random access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, Calif. and Motorola Corporation of Schaumburg, Ill. Such processors include, or may be in communication with, media, for example computer-readable media, which stores instructions that, when executed by the processor, cause the processor to perform the steps described herein.

Embodiments of computer-readable media can include an electronic, optical, magnetic, or other storage or transmission device capable of providing a processor with computer-readable instructions. Other examples of suitable media include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. The instructions may comprise code from any suitable computer-programming language, including, for example, C, C++, C#, Visual Basic, Java, Python, Perl, and JavaScript.

Computers are connected in some embodiments to a network. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers related to aspects of the technology provided herein may be any type of processor-based platform that operates on any operating system, such as Microsoft Windows, Linux, UNIX, Mac OS X, etc., capable of supporting one or more programs comprising the technology provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device.

All such components, computers, and systems described herein as associated with the technology may be logical or virtual.

It is also envisioned that embodiments could be accomplished as computer signals embodied in a carrier wave, as well as signals (e.g., electrical and optical) propagated through a transmission medium. Thus, the various types of information discussed above could be formatted in a structure, such as a data structure, and transmitted as an electrical signal through a transmission medium or stored on a computer readable medium.

In some embodiments, the disclosure provides a system for predicting progression of breast cancer. The breast cancer can be one or more of ductal carcinoma in situ, invasive ductal carcinoma, inflammatory breast cancer and metastatic breast cancer. In an embodiment, a breast cancer can be identified and the breast cancer predicted in an individual, the system comprising: an apparatus configured to determine expression levels of nucleic acids, proteins, peptides or other molecule from a biological sample taken from the individual; and hardware logic designed or configured to perform operations comprising: (a) receiving expression levels of a collection of signature genes from a biological sample taken from said individual, wherein said collection of signature genes comprises at least two genes selected from the sequences set forth in Table 1.

Information relevant to the patient's diagnosis include, but are not limited to, age, ethnicity, tumor localization, pertinent past medical history related to co-morbidity, other oncological history, family history for cancer, physical exam findings, radiological findings, biopsy date, biopsy result, types of operation performed (radical retropubic or radical perineal prostatectomy), neoadjuvant therapy (i.e. chemotherapy, hormones), adjuvant or salvage radiotherapy, hormonal therapy, local vs. distant disease recurrence and survival outcome. These clinical variables may be included in the predictive model in various embodiments.

Once a biomarker or biomarker panel is selected, a method for diagnosing an individual that may be suffering from a breast cancer. In an embodiment, a biomarker or biomarker panel is selected, a method for diagnosing an individual that may be suffering from a breast cancer and can comprise one or more of the following steps: 1) collect or otherwise obtain a biological sample; 2) perform an analytical method to detect and measure the biomarker or biomarkers in the panel in the biological sample; 3) perform any data normalization or standardization required for the method used to collect biomarker values; 4) calculate a biomarker score; 5) combine the biomarker scores to obtain a total diagnostic score; and 6) report the individual's diagnostic score. This method of diagnosis can be conducted using a computer and software programs for analysis of data collected from nucleic acid, protein, peptide or other biological molecules. In this approach, the diagnostic score may be a single number determined from the sum of all the marker calculations that is compared to a preset threshold value that is an indication of the presence or absence of disease. Or the diagnostic score may be a series of bars that each represent a biomarker value and the pattern of the responses may be compared to a pre-set pattern for determination of the presence or absence of disease.

For both DNA and RNA, the nucleic acid can be isolated from a saliva, plasma or blood sample. The DNA or RNA can be extracellular or extracted from a cell in the plasma or blood sample. The DNA or RNA can also be extracted from a cellular biopsy, including from a tumor, including, a solid tumor in the breast.

For a protein or peptide or other biological molecule, such can be isolated from a saliva, plasma or a blood sample. The protein or peptide or other biological molecule can be extracellular or extracted from a cell in the saliva, plasma or a blood sample. The protein or peptide or other biological molecule can also be extracted from a cellular biopsy, including from a tumor, including, a solid tumor in the breast.

It is also noted that many of the structures, materials, and acts recited herein can be recited as means for performing a function or step for performing a function. Therefore, it should be understood that such language is entitled to cover all such structures, materials, or acts disclosed within this specification and their equivalents, including the matter incorporated by reference.

The breast cancer biomarker analysis system can provide functions and operations to complete data analysis, such as data gathering, processing, analysis, reporting and/or diagnosis. For example, in one embodiment, the computer system can execute the computer program that may receive, store, search, analyze, and report information relating to the biomarkers. The computer program may comprise multiple modules performing various functions or operations, such as a processing module for processing raw data and generating supplemental data and an analysis module for analyzing raw data and supplemental data to generate an breast cancer status and/or diagnosis. Diagnosing the status of breast cancer may comprise generating or collecting any other information, including additional biomedical information, regarding the condition of the individual relative to the disease, identifying whether further tests may be desirable, or otherwise evaluating the health status of the individual.

The breast cancer biomarker analysis system can provide functions and operations to complete data analysis, such as data gathering, processing, analysis, reporting and/or diagnosis. For example, in one embodiment, the computer system can execute the computer program that may receive, store, search, analyze, and report information relating to the breast cancer biomarkers. The computer program may comprise multiple modules performing various functions or operations, such as a processing module for processing raw data and generating supplemental data and an analysis module for analyzing raw data and supplemental data to generate a breast cancer status and/or diagnosis. Diagnosing breast cancer status may comprise generating or collecting any other information, including additional biomedical information, regarding the condition of the individual relative to the disease, identifying whether further tests may be desirable, or otherwise evaluating the health status of the individual.

As used herein, a “computer program product” refers to an organized set of instructions in the form of natural or programming language statements that are contained on a physical media of any nature (e.g., written, electronic, magnetic, optical or otherwise) and that may be used with a computer or other automated data processing system. Such programming language statements, when executed by a computer or data processing system, cause the computer or data processing system to act in accordance with the particular content of the statements. Computer program products include without limitation: programs in source and object code and/or test or data libraries embedded in a computer readable medium. Furthermore, the computer program product that enables a computer system or data processing equipment device to act in pre-selected ways may be provided in a number of forms, including, but not limited to, original source code, assembly code, object code, machine language, encrypted or compressed versions of the foregoing and any and all equivalents.

In one embodiment, a computer program product is provided for indicating a likelihood of breast cancer. The computer program product includes a computer readable medium embodying program code executable by a processor of a computing device or system, the program code comprising: code that retrieves data attributed to a biological sample from an individual, wherein the data comprises biomarker values that each correspond to one of at least N biomarkers in the biological sample selected from the group of biomarkers provided in Table 1; and code that executes a classification method that indicates a breast cancer status of the individual as a function of the biomarker values.

In yet another embodiment, a computer program product is provided for indicating a likelihood of breast cancer. The computer program product includes a computer readable medium embodying program code executable by a processor of a computing device or system, the program code comprising: code that retrieves data attributed to a biological sample from an individual, wherein the data comprises a biomarker value corresponding to a biomarker in the biological sample selected from the group of biomarkers provided in Table 1; and code that executes a classification method that indicates a breast cancer status of the individual as a function of the biomarker value.

The kit (i.e., diagnostic kit) can include reagents for determining, from a plasma sample of a subject, the amount of mRNAs or mutations in a gene based on assaying the nucleic acids, proteins, peptides or other biological molecule isolated from a breast cancer, a circulating cell or the remnants of a circulating cell present in plasma, including a protein, peptide or other biological molecule. The nucleic acid can be a deoxyribonucleic acid (DNA), a ribonucleic acid (RNA) and/or an artificial nucleic acid, including an artificial nucleic acid analogue. Along with mRNAs, other RNAs include non-coding RNA (ncRNA), transfer RNA (tRNA), messenger RNA (mRNA), small interfering RNA (siRNA), piwi RNA (piRNA), small nuclear RNA (snoRNA), small nuclear (snRNA), extracellular RNA (exRNA), and ribosomal RNA (rRNA).

The disclosed methods and assays provide for convenient, efficient, and potentially cost-effective means to obtain data and information useful in assessing appropriate or effective therapies for treating patients. The kit can use conventional methods for detecting the biomarkers, whether a protein, peptide, other biological molecule or an RNA or a DNA to be assessed include protocols that examine the presence and/or expression of a desired nucleic acid, for example a SNP, in a sample. Tissue or cell samples from mammals can be conveniently assayed for, e.g., genetic-marker RNA, including in an embodiment an mRNA or DNAs using Northern, dot-blot, or polymerase chain reaction (PCR) analysis, array hybridization, RNase protection assay, or using DNA SNP chip microarrays, which are commercially available, including DNA micro array snapshots. For example, real-time PCR (RT-PCR) assays such as quantitative PCR assays are well known in the art.

Probes used for PCR can be labeled with a detectable marker, such as, for example, a radioisotope, fluorescent compound, bioluminescent compound, a chemiluminescent compound, metal chelator, or enzyme. Such probes and primers can be used to detect the presence of a mutation in a DNA, an RNA and in one embodiment, an mRNA in a sample and as a means for detecting a cell expressing the mRNA. As will be understood by the skilled artisan, a great many different primers and probes can be prepared based on known sequences and used effectively to amplify, clone, and/or determine the presence and/or levels of mRNAs.

Other DNA tests to determine whether a mutation exists include fluorescence in situ hybridization (FISH). Through the use of FISH, a mutation can be detected in cells, tissues, including breast tissue, including tumors, and further including breast tumors.

Other methods include protocols that examine or detect a mutation in a DNA or an RNA. These other methods include protocols that examine or detect mRNAs in a tissue or cell sample by microarray technologies. Using nucleic acid microarrays, test and control RNAs, including in an embodiment, mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes that have potential to be expressed in certain disease states can be arrayed on a solid support. Hybridization of a labeled probe with a particular array member indicates that the sample from which the probe was derived expresses that gene. Differential gene expression analysis of disease tissue can provide valuable information. Microarray technology utilizes nucleic acid hybridization techniques and computing technology to evaluate the mRNA expression profile of thousands of genes within a single experiment.

The biomarkers are particularly useful in cancer diagnosis as their expression patterns is different when comparing healthy subjects with subjects that have breast cancer. The expression of biomarkers typically includes both up- and down-regulated levels of mRNAs. In an embodiment, the biomarkers set forth herein can determine if a patient has breast cancer or does not have breast cancer. In an embodiment, each is a form of breast cancer (e.g., ductal carcinoma in situ, invasive ductal carcinoma, inflammatory breast cancer or metastatic breast cancer).

The biomarkers below can be detected in DNA, including in an embodiment, one or mutations associated with a region of a gene, a snp or one or mutation on one or more chromosomes. The biomarkers below can also be detected in an RNA, including in an embodiment, an miRNA, a tRNA, an mRNA or other form of RNA.

mRNA as Biomarkers

Table 1 includes a list of mRNA biomarkers for detecting breast cancer.

TABLE 1
mRNA Biomarkers for Breast Cancer

| No. | Probe ID | Gene Symbol | Gene Name |
|---|---|---|---|
| 1 | 1556583_a_at | SLC8A1 | Solute Carrier Family 8 |
| | | | Member A1 |
| 2 | 1560982_at | RP11-452L6.1 | — |
| 3 | 228697_at | HINT3 | Histidine Triad Nucleotide |
| | | | Binding Protein 3 |
| 4 | 237291_at | PRORSD1P | Prolyl-TRNA Synthetase |
| | | | Associated Domain |
| | | | Containing 1, Pseudogene |
| 5 | 242751_at | PRDX6 | Peroxiredoxin 6 |
| 6 | 1557122_s_at | GABRB2 | Gamma-Aminobutyric Acid |
| | | | Type A |
| | | | Receptor Subunit Beta2 |
| 7 | 202609_at | EPS8 | Epidermal Growth Factor |
| | | | Receptor |
| | | | Pathway Substrate 8 |
| 8 | 225590_at | SH3RF1 | SH3 Domain Containing |
| | | | Ring Finger 1 |
| 9 | 235757_at | GB_ACC AA814006 | — |
| 10 | 238774_at | Hypothetical protein | KANSL1 |
| | | LOC284058 | |
| 11 | 239954_at | ZNF160 | Zinc Finger Protein 160 |
| 12 | 1563498_s_at | SLC25A45 | Solute Carrier Family 25 |
| | | | Member 45 |
| 13 | 212254_s_at | DST | Dystonin |
| 14 | 231984_at | MTAP | Methylthioadenosine |
| | | | Phosphorylase |
| 15 | 234326_at | GB_Acc AK024901 | — |
| 16 | 232022_at | TFB1M | Transcription Factor B1, |
| | | | Mitochondrial |
| 17 | 226837_at | SPRED1 | Sprouty Related EVH1 |
| | | | Domain Containing 1 |
| 18 | 212097_at | CAV1 | Caveolin-1 |
| 19 | 213693_s_at | MUC1 | Mucin 1, also |
| | | | called polymorphic |
| | | | epithelial mucin or EMA |
| 20 | 204749_at | NAP1L3 | Nucleosome assembly protein |
| | | | 1 like 3 |
| 21 | 238721_at | MDH1B | Putative malate |
| | | | dehydrogenase 1B |
| 22 | 225822_at | TMEM125 | Transmembrane protein 125 |
| 23 | 205593_s_at | PDE9A | Phosphodiesterease 9A |
| 24 | 235616_at | TSHZ2 | Teashirt Zinc Finger |
| | | | Homeobox 2 |
| 25 | 234334_s_at | LAMB4 | Laminin Subunit Beta 4 |
| 26 | 1554997_a_at | PTGS2 | Prostaglandin-endoperoxide |
| | | | synthase 2 |

_at=all the probes hit one known transcript;
_a=all probes in the set hit alternate transcripts from the same gene;
_s=all probes in the set hit transcripts from different genes;
_x=some probes hit transcripts from different genes.

Methods of determining the level of the biomarker besides RT-PCR or another PCR-based method include proteomics techniques, as well as individualized genetic profiles. Individualized genetic profiles can be used to treat RA based on patient response at a molecular level. The specialized microarrays herein, (e.g., oligonucleotide microarrays or cDNA microarrays) can include one or more biomarkers having expression profiles that correlate with either sensitivity or resistance to one or more antibodies. While a single biomarker can provide evidence of breast cancer, reliability and accuracy is improved when multiple biomarkers are used in the methods described herein.

Method of Breast Cancer Diagnoses Using mRNAs as Biomarkers

Detection and quantification of expressed mRNA can use standard techniques known to one skilled in the art. For example, the expression or amount of a particular mRNA in the body fluid sample or in the tissue specimen, or in a breast biopsy sample or a breast tissue sample identified, is determined by immunohistochemical (INC) methods, by immunofluorescence (IF) methods, by RNA in-situ hybridization, by reverse transcriptase polymerase chain reaction (RTPCR), especially quantitative real time RT-PCR (qRT-PCR), or by a combination of these methods. Other methods for determining the level of (expression of) a particular mRNA include MALDI-MS (including surface enhanced laser desorption/ionization mass spectrometry (SELDI-MS), especially surface-enhanced affinity capture (SEAC), surface-enhanced need desorption (SEND) or surface-enhanced photo label attachment and release (SEPAR), antibody testing (including immunoprecipitation, Western blotting, Enzyme-linked immune-sorbent assay (ELISA), Enzyme-linked immuno sorbent assay (RIA), dissociation-enhanced lanthanide fluoro-immunoassay (DELFIA), scintillation proximity assay (SPA), and quantitative nucleic acid testing, especially PCR, LCR and RT-PCR of samples for marker (KRT23) mRNA detection and quantification.

Preferably the difference in protein amount or in expressed mRNA for a biomarker is at least 5%, 10% 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 165%, 170%, 180%, 190%, 200%, 210%, 220%, 230%, 240%, 250%, 260%, 270%, 280%, 290%, 300%, 325%, 350%, 375%, or 400%. Preferably the difference in protein amount or in expressed mRNA for a biomarker is, more preferred at least 50% or may even be as high as 75% or 100%. More preferred this difference in the level of expression or protein amount is at least 200%, i.e., two-fold, at least 500%, i.e., five-fold, or at least 1000%, i.e., 10-fold. The expression level for a biomarker according to the present invention expressed lower or higher in a breast cancer cell sample than in a healthy, normal breast sample is at least 5%, 10% or 20%, more preferred at least 50% or may even be 75% or 100%, i.e., two-fold higher, preferably at least ten-fold higher in the breast cancer cell sample. Whether a biomarker level is increased in a given detection method can be established by analysis of a multitude of disease samples with the given detection method. This can then form a suitable level from which the “increased” status can be determined; e.g., by the above % difference or -fold change. A changed level of expression of a biomarker can be indicative of breast cancer. In some cases, no biomarker expression is detectable in healthy samples. In such case, any detection with normal test systems is already an “increased” level within the meaning of the present application.

One or more of the biomarkers can be used in a method of diagnosing cancer or determining a prognosis of a test subject with breast cancer. In this manner, one biomarker or a combination of 2, 3, 4, 5, 6, 7, 8, 9, 10, 11, 12, 13, 14, 15, 16, 17, 18, 19, 20, 21, 22, 23, 24, 25 or 26 biomarkers can be used in a method of diagnosing breast cancer or determining a prognosis of a test subject with breast cancer. In this manner, at least one biomarker or a combination of at least 2, 3, 4, 5, 6, 7, 8, 9, 10, 11, 12, 13, 14, 15, 16, 17, 18, 19, 20, 21, 22, 23, 24, 25 or 26 biomarkers can be used in a method of diagnosing cancer or determining a prognosis of a test subject with breast cancer.

In this manner, no more than one biomarker or a combination of no more than 2, 3, 4, 5, 6, 7, 8, 9, 10, 11, 12, 13, 14, 15, 16, 17, 18, 19, 20, 21, 22, 23, 24, 25 or 26 biomarkers can be used in a method of diagnosing breast cancer or determining a prognosis of a test subject with cancer. In this manner, about one biomarker or a combination of about 2, 3, 4, 5, 6, 7, 8, 9, 10, 11, 12, 13, 14, 15, 16, 17, 18, 19, 20, 21, 22, 23, 24, 25 or 26 biomarkers can be used in a method of diagnosing breast cancer or determining a prognosis of a test subject with breast cancer.

In a first step, the expression levels of one or more nucleic acids, including DNA and/or RNA (e.g., mRNA) are measured in saliva, plasma, blood or tissue samples from healthy subjects samples from subjects with cancer. In an embodiment, the expression levels of one biomarker or a combination of 2, 3, 4, 5, 6, 7, 8, 9, 10, 11, 12, 13, 14, 15, 16, 17, 18, 19, 20, 21, 22, 23, 24, 25 or 26 biomarkers can be used to generate a footprint or signature for subsequent diagnosis of patients. In an embodiment, the expression levels of at least one biomarker or a combination of at least 2, 3, 4, 5, 6, 7, 8, 9, 10, 11, 12, 13, 14, 15, 16, 17, 18, 19, 20, 21, 22, 23, 24, 25 or 26 biomarkers can be used to generate a footprint or signature for subsequent diagnosis of patients.

In an embodiment, the expression levels of no more than one biomarker or a combination of no more than 2, 3, 4, 5, 6, 7, 8, 9, 10, 11, 12, 13, 14, 15, 16, 17, 18, 19, 20, 21, 22, 23, 24, 25 or 26 biomarkers can be used to generate a footprint or signature for subsequent diagnosis of patients. In an embodiment, the expression levels of about one biomarker or a combination of about 2, 3, 4, 5, 6, 7, 8, 9, 10, 11, 12, 13, 14, 15, 16, 17, 18, 19, 20, 21, 22, 23, 24, 25 or 26 biomarkers can be used to generate a footprint or signature for subsequent diagnosis of patients.

Next, expression levels of the same nucleic acids, including DNA and/or RNA (e.g., mRNA) are measured in saliva, plasma, blood or tissue samples from healthy subjects. This is used as a control. Thereafter, samples from healthy patients can be compared to identifying mRNAs that have altered levels of expression in the plasma samples from the subjects with cancer. A biomarker fingerprint or signature can be created from the mRNAs with altered levels of expression. This can be used for diagnosing or determining the prognosis of cancer in the test subject by comparing of levels of mRNAs from plasma of the test subject. Conventional statistical analysis can be used to determine, for example, confidence levels.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

### Predictive Models

According to some embodiments of the subject technology, combinations of small numbers of markers (four or fewer) have been determined to discriminate response with high accuracy based on a data set of studies relating to breast cancer. The markers have been validated against data sets as described below.

### Example 1

### Discovery of Biomarkers

GSE27567 Dataset—Serum from Peripheral Blood

A published data set was used to discover biomarkers that are predictive or indicative of the presence of breast cancer. The study included 72 patients with positive breast cancer biopsies and 68 patients with negative breast cancer biopsies. In total, 54,613 mRNA biomarkers were measured from peripheral blood mononuclear cells. Patient data was randomized equally across three subgroups: training, selection, and out-of-sample test groups (preserving proportions of the two diagnostic classes).

The study considered all 54,613 variables using Applicant's mathematical evolution modeling platform (see. e.g., U.S. Pat. No. 9,845,505). The published study is cited as LaBreche H G, et. al, “Integrating factor analysis and a transgenic mouse model to reveal a peripheral blood predictor of breast tumors,” BMC Med Genomics. 2011 Jul. 22; 4:61. doi: 10.1186/1755-8794-4-61. PMID: 21781289; PMCID: PMC3178481. The data set and Affymetrix Human Genome U133 Plus 2.0 Array can be found at the ncbi website (NCBI>GEO>Accession Display; Platform GPL570 and Platform GSE27567)

Seventeen biomarkers were identified as: (1) SLC8A1, (2) RP11-452L6.1, (3) HINT3, (4) PRORSD1P, (5) PRDX6, (6) GABRB2, (7) EPS8, (8) SH3RF1, (9) GB_ACC AA814006, (10) Hypothetical protein LOC284058, (11) ZNF160, (12) SLC25A45, (13) DST, (14) MTAP, (15) GB_Acc AK024901, (16) TFB1M and (17) SPRED1. In order to validate the sets of markers, Applicants obtained four additional data sets of gene expression markers for breast cancer patients. These data sets are discussed in the following examples:

### Example 2

### Validation of Biomarkers

### GSE20266 Dataset—Saliva

A published data set was used to validate the effectiveness of the biomarkers as predictive or indicative of the presence of breast cancer. The study included 10 patients with positive breast cancer biopsies and 10 patients with negative breast cancer biopsies. In total, 54,613 mRNA biomarkers were measured from peripheral blood mononuclear cells. Patient data was randomized equally across three subgroups: training, selection, and out-of-sample test groups (preserving proportions of the two diagnostic classes).

As above, the study considered 54,613 variables using Applicant's mathematical evolution modeling platform. The published study is cited as LaBreche H G, et. al, “Integrating factor analysis and a transgenic mouse model to reveal a peripheral blood predictor of breast tumors,” BMC Med Genomics. 2011 Jul. 22; 4:61. doi: 10.1186/1755-8794-4-61. PMID: 21781289; PMCID: PMC3178481. The data set and Affymetrix Human Genome U133 Plus 2.0 Array can be found at the ncbi website (NCBI>GEO>Accession Display; Platform GPL570 and Platform GSE27567)

### Example 3

### Validation of Biomarkers

### GSE47860 Dataset, Ontario Canada Cohort—Blood

A second published data set was used to validate the effectiveness of the biomarkers as predictive or indicative of the presence of breast cancer. The study included 21 patients with positive breast cancer biopsies and 15 patients with negative breast cancer biopsies. In total, 54,613 mRNA biomarkers were measured from peripheral blood mononuclear cells. Randomize patients equally across three subgroups: training, selection, and out-of-sample test groups (preserving proportions of the two diagnostic classes).

As above, the study considered 38,079 mRNA biomarkers obtained from peripheral blood mononuclear cells. The published study is cited as Piccolo S R, et. al, “Integrative analyses reveal signaling pathways underlying familial breast cancer susceptibility,” Mol Syst Biol. 2016 Mar. 10; 12(3):860. doi: 10.15252/msb.20156506. PMID: 26969729; PMCID: PMC4812528. The data set and Affymetrix Human Genome U133 Plus 2.0 Array can be found at the ncbi website (NCBI>GEO>Accession Display; Platform GSE47860 and Platform GPL12729).

### Example 4

### Validation of Biomarkers

### GSE47860 Dataset, Utah Cohort—Blood

A third published data set was used to validate the effectiveness of the biomarkers as predictive or indicative of the presence of breast cancer. The study included 61 patients with positive breast cancer biopsies and 63 patients with negative breast cancer biopsies. In total, 54,613 mRNA biomarkers were measured from peripheral blood mononuclear cells. Randomize patients equally across three subgroups: training, selection, and out-of-sample test groups (preserving proportions of the two diagnostic classes).

As above, the study considered 38,079 mRNA biomarkers obtained from peripheral blood mononuclear cells. The published study is cited as Piccolo S R, et. al, “Integrative analyses reveal signaling pathways underlying familial breast cancer susceptibility,” Mol Syst Biol. 2016 Mar. 10; 12(3):860. doi: 10.15252/msb.20156506. PMID: 26969729; PMCID: PMC4812528. The data set and Affymetrix Human Genome U133 Plus 2.0 Array can be found at the ncbi website (NCBI>GEO>Accession Display; Platform GSE47860 and Platform GPL17279).

### Example 5

### Validation of Biomarkers

### GSE47861 Dataset, Ontario Cohort—Blood

A fourth published data set was used to validate the effectiveness of the biomarkers as predictive or indicative of the presence of breast cancer. The study included 15 patients with positive breast cancer biopsies and 22 patients with negative breast cancer biopsies. In total, 54,613 mRNA biomarkers were measured from peripheral blood mononuclear cells. Randomize patients equally across three subgroups: training, selection, and out-of-sample test groups (preserving proportions of the two diagnostic classes).

As above, the study considered 38,079 mRNA biomarkers obtained from peripheral blood mononuclear cells. The published study is cited as Piccolo S R, et. al, “Integrative analyses reveal signaling pathways underlying familial breast cancer susceptibility,” Mol Syst Biol. 2016 Mar. 10; 12(3):860. doi: 10.15252/msb.20156506. PMID: 26969729; PMCID: PMC4812528. The data set and Affymetrix Human Genome U133 Plus 2.0 Array can be found at the ncbi website (NCBI>GEO>Accession Display; Platform GSE47860 and Platform GPL17279).

The results of the discovery and validation studies are summarized in Table 2. The positive and negative predictive values (PPV and NPV respectively) are the proportions of positive and negative results in statistics and diagnostic tests that are true positive and true negative results, respectively.

TABLE 2
Summary of Discovery/Validation Studies

| | | Validation | Validation | Validation |
|---|---|---|---|---|
| Discovery | Validation | GSE47860 | GSE47860 | GSE47861 |
| GSE27567 | GSE20266 | Ontario | Utah | Ontario |
| (Blood) | (Saliva) | (Blood) | (Blood) | (Blood) |
| Under 40 Model | Under 40 Model | Under 40 Model | Under 40 Model | Under 40 Model |
| Sensitivity 93% | Sensitivity 100% | Sensitivity 100% | Sensitivity 87% | Sensitivity 100% |
| Specificity 97% | Specificity 100% | Specificity 100% | Specificity 97% | Specificity 100% |
| PPV 56.4% | PPV 100% | PPV 100% | PPV 54.7% | PPV 100% |
| NPV 99.7% | NPV 100% | NPV 100% | NPV 99.4% | NPV 100% |
| (4% prevalence) | (4% prevalence) | (4% prevalence) | (4% prevalence) | (4% prevalence) |
| Over 55 Model | Over 55 Model | Over 55 Model | Over 55 Model | Over 55 Model |
| Sensitivity 97% | Sensitivity 100% | Sensitivity 100% | Sensitivity 95% | Sensitivity 100% |
| Specificity 87% | Specificity 100% | Specificity 100% | Specificity 94% | Specificity 100% |
| PPV 65.1% | PPV 100% | PPV 100% | PPV 79.8% | PPV 100% |
| NPV 99.1% | NPV 100% | NPV 100% | NPV 98.7% | NPV 100% |
| (20% prevalence) | (20% prevalence) | (20% prevalence) | (20% prevalence) | (20% prevalence) |

### Example 6

### Discovery of Additional Biomarkers

GSE27567 Dataset—Serum from Peripheral Blood

Applicants pursued additional markers by conducting a second discovery study. The same discovery and validation datasets were used as described above using the following approach:
1) Configure Emerge to ignore the original 17 biomarkers and consider the remaining 54,596 gene expression probes;
2) Produce a binary classifier to distinguish between patients with a confirmed positive breast cancer biopsy and patients with a confirmed negative breast cancer biopsy;
3) Identify the first minimal set of biomarkers that maximizes diagnostic accuracy; 4) Set aside the first minimal set and repeat the analysis with the remaining biomarkers, to determine the next minimal set; and
5) Repeat until observing a significant decline in diagnostic accuracy.

Two sets of biomarkers were obtained (i.e., group 1 and group 2) which were validated as described above. Group 1 biomarkers were identified as: (18) CAV1, (19) MUC1, (20) NAP1L3, (21) MDH1B, (22) TMEM125, (23) PDE9A. Group 2 biomarkers were identified as: (24) TSHZ2, (25) LAMB4 and (26) PTGS2. The results of the discovery and validation studies are summarized in Table 3.

TABLE 3
Summary of Discovery/Validation Studies

| | | | Validation | Validation | Validation |
|---|---|---|---|---|---|
| | Discovery | Validation | GSE47860 | GSE47860 | GSE47861 |
| | GSE27567 | GSE20266 | Ontario | Utah | Ontario |
| | (Blood) | (Saliva) | (Blood) | (Blood) | (Blood) |
| Group 1 | Sensitivity 72% | Sensitivity 93% | Sensitivity 91% | Sensitivity 74% | Sensitivity 88% |
| | Specificity 71% | Specificity 92% | Specificity 94% | Specificity 79% | Specificity 87% |
| Group 2 | Sensitivity 68% | Sensitivity 68% | Sensitivity 76% | Sensitivity 74% | Sensitivity 89% |
| | Specificity 69% | Specificity 69% | Specificity 100% | Specificity 75% | Specificity 73% |

### Example 7

### Early Detection of Breast Cancer Using Biomarkers

A pre-symptomatic diagnosis of patients with breast cancer would be of great value, not only for a better understanding of the disease's pathophysiology, but also for providing early treatment and mitigation efforts. In this example, a patient has an early-stage breast cancer that is not identifiable through manual manipulation using a hand to identify a lump in the breast. The patient is a 50-year-old female who desires to be evaluated by a physician for the possibility of breast cancer. The patient is borderline obese and has a history of alcohol abuse and high blood pressure. The patient also has a family history of breast cancer. Because of these risk factors, the physician orders a mammogram.

The result or the mammogram is that no identifiable lump is detected by some irregular cell growth may have been seen. The patient is then screened using the biomarkers of Table 1. The patient provides a sample (e.g., blood, plasma, urine or saliva) for biomarker analysis. The results indicate that the patient has an early stages of breast cancer. Based on these results, the patient is referred to specialist for further evaluation and possible therapeutic treatment. The physician also recommends regular (i.e., quarterly, biannual or annual) testing to ensure that the breast cancer does not advance.

### Example 8

### Detection of Breast Cancer Following Identification of a Lump in the Breast Using Biomarkers

In this example, a patient goes to her doctor after identifying an unusual lump in her left breast. Her doctor orders her to get a mammogram, which she has done, and a lump is seen in her left breast.

In this example, confirmation of a diagnosis of breast cancer is desired. To this end, the physician orders a biopsy be taken from the site of the suspected tumor in the patient's breast. Following removal of the biopsy sample, the biomarker test described herein using one or more of the seventeen biomarkers identified in Table 1 is conducted to confirm the presence of the breast cancer and determine the progress of the breast cancer. The patient provides a sample for biomarker analysis. Specifically, the test determines progression from benign to metastatic cancer. The test also identifies the type of breast cancer and in genetic type of the breast cancer.

The test indicates that the patient has breast cancer. As with the first example, the patient is referred to specialist for further evaluation and the specialist will then determine the course of treatment and therapeutics to be used and the treatment protocol. The physician also recommends regular (i.e., quarterly, biannual or annual) testing to ensure that the breast cancer does not advance.

### Example 9

### Detection of Breast Cancer Using Biomarkers (Asymptomatic)

In this example, a patient has no symptoms of breast cancer but undergoes evaluation because of known genetic risk factors. A manual manipulation of the breast using the doctor's hands and a mammogram do not show a breast cancer tumor. However, the patient complains of soreness of the breast.

The physician requests a test of the biomarkers test described herein (Table 1) to detect/determine a prognosis of breast cancer. The patient provides a sample for biomarker analysis. The test provides the physician with biomarker levels which indicates the presence of early-stage breast cancer. As with the first example, the patient is referred to a specialist for further evaluation and to discuss treatment options.

Based on the progress of the breast cancer, the specialist can devise a treatment plan. Medical treatments may be necessary to remove, shrink, or slow the growth of tumors. Typically, treatment is based on the type of breast cancer and its stage. Other factors, including the patient's overall health, menopause status and personal preferences. Common treatments include surgery, radiation, chemotherapy, hormone therapy, targeted drug therapy and immunotherapy. Drugs (i.e., systemic therapies) can be administered to treat breast cancer. Common targeted drugs include as trastuzumab (HERCEPTIN™), pertuzumab (PERJETA™), or abemaciclib (VERZENIO™). The most common form of treatment for breast cancer is surgery. This involves removing the tumor and nearby margins. Surgical options may include a lumpectomy, partial mastectomy, radical mastectomy, and reconstruction.

In this example, the specialist suggests a combination of hormone therapy (i.e., TAMOXIFEN™, an aromatase inhibitor) and a biopsy of the suspect tissue. The physician also recommends regular (i.e., quarterly) testing to ensure that the breast cancer does not advance.

### Example 10

### Detection of Breast Cancer Using Biomarkers (Asymptomatic, Genetic Risk Factor)

In this example, a patient has no symptoms of breast cancer but undergoes evaluation because of known genetic risk factors. A manual manipulation of the breast using the doctor's hands and a mammogram find a lump that the doctor suspects is a breast cancer tumor. The doctor also suspects that the breast cancer has metastasized.

The physician collects a blood sample from the patient and orders the use of one or more of the biomarkers identified in Table 1 to determine if the breast cancer has metastasized (as determined by the presence of circulating breast cancer cells). The test provides the physician with biomarker levels to diagnose the patient with breast cancer cells in the blood suggesting that the cancer has metastasized. The test also identifies the type of breast cancer and in the genetic type of the breast cancer. As with the first example, the patient is referred to specialist for further evaluation and to discuss treatment options.

In this example, the specialist suggests a combination of surgery and targeted drug therapy. The physician also recommends regular (i.e., quarterly) testing to monitor progression of the cancer.

### Example 11

### Detection of Breast Cancer Using Biomarkers (Asymptomatic, Genetic Risk Factor)

In this example, a patient has no symptoms of breast cancer but undergoes evaluation because of known genetic risk factors. A manual manipulation of the breast using the doctor's hands and a mammogram do not show a breast cancer tumor. However, the patient complains of soreness of the breast.

The physician has a biopsy of the patient's tissue where the tumor is located. The doctor orders that a subset of the biomarkers found in Table 1 be used to determine the presence of a breast cancer. To confirm the result, the doctor orders a second test with the remaining biomarkers not used in the first test. The patient provides a sample for biomarker analysis. The test provides the physician with biomarker levels that are sufficient to diagnose the patient with breast cancer and to confirm the result with the second test. As with the first example, the patient is then referred to specialist for further evaluation and to discuss treatment options.

### Example 12

### Diagnostic Kit for Rapid Screening of Breast Cancer

The following working example is based on configurations described above. Embodiments of the invention can be compiled into a diagnostic kit for diagnosing breast cancer. The kit can identify one or more target cells that have the biomarkers for breast cancer in plasma from a test subject.

The kit can include a collection of nucleic acid molecules such that each nucleic acid molecule encodes a mRNA sequence. The nucleic acid molecules can be used to identify variations in expression levels of one or more mRNAs in a plasma sample from a test subject. The expression levels of the mRNAs can be used in a comparison/analysis of test samples with a fingerprint indicative of the presence of cancer.

In certain embodiments, the present disclosure provides kits for diagnosing breast cancer. The kits can include one biomarker or a combination of 2, 3, 4, 5, 6, 7, 8, 9, 10, 11, 12, 13, 14, 15, 16, 17, 18, 19, 20, 21, 22, 23, 24, 25 or 26 biomarkers disclosed herein. The skilled artisan will appreciate that the number of biomarkers may be varied without departing from the nature of the present disclosure, and thus other combinations of biomarkers are also encompassed by the present disclosure. The skilled artisan will know which one biomarker or a combination of 2, 3, 4, 5, 6, 7, 8, 9, 10, 11, 12, 13, 14, 15, 16, 17, 18, 19, 20, 21, 22, 23, 24, 25 or 26 biomarkers to use based on the symptoms of the patient suffering from breast cancer.

In a specific embodiment, a kit includes the one biomarker or a combination of 2, 3, 4, 5, 6, 7, 8, 9, 10, 11, 12, 13, 14, 15, 16, 17, 18, 19, 20, 21, 22, 23, 24, 25 or 26 biomarkers disclosed herein. In certain embodiments, the kit is for diagnosing breast cancer. The kit can further optionally include instructions for use. The kit can further optionally include (e.g., comprise, consist essentially of, consist of) tubes, applicators, vials or other storage container with the above-mentioned biomarker and/or vials containing one or more of the biomarkers. In an embodiment, each biomarker is in its own tube, applicator, vial or storage container or 2, 3, 4, 5, 6, 7, 8, 9, 10, 11, 12, 13, 14, 15, 16, 17, 18, 19, 20, 21, 22, 23, 24, 25 or 26 biomarkers are in a tube, applicator, vial or storage container.

The kits, regardless of type, will generally include one or more containers into which the one biomarker or a combination of 2, 3, 4, 5, 6, 7, 8, 9, 10, 11, 12, 13, 14, 15, 16, 17, 18, 19, 20, 21, 22, 23, 24, 25 or 26 biomarkers are placed and, preferably, suitably aliquoted. The components of the kits may be packaged either in aqueous media or in lyophilized form.

### Example 13

### Screening a Patient for Breast Cancer

A female smoker, age 56, presents to her doctor with a family history of breast cancer but no noticeable lumps or physical manifestations in her breasts. The doctor draws a sample of blood and sends it to a lab to test for breast cancer. The blood sample is prepared and the plasma is obtained. The plasma is then tested to identify the presence of biomarkers associated with breast cancer. The lab uses one or more of the following biomarkers in its test: SLC8A1, RP11-452L6.1, HINT3, PRORSD1P, PRDX6, GABRB2, EPS8, SH3RF1, GB_ACC AA814006, Hypothetical protein LOC284058, ZNF160, SLC25A45, DST, MTAP, GB_Acc AK024901, TFB1M, SPRED1, CAV1, MUC1, NAP1L3, MDH1B, TMEM125, PDE9A, TSHZ2, LAMB4 and/or PTGS2. Following the test, the lab determines that the one or more biomarkers used to test for breast cancer are indicative of the presence of cancer. The lab sends the test results to the doctor. Upon a subsequent visit to the doctor, the patient is informed of the result. The patient is examined thoroughly (including with a mammogram) and the doctor removes a sample of the suspect tissue for biopsy.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

What is claimed is:

1. A method of detecting a breast cancer or determining a prognosis of a subject with a breast cancer, comprising the steps of:
a) measuring the expression level of at least one mRNA in a test sample from the subject,
b) receiving the expression level of the at least one mRNA in the test sample by a computer and
c) comparing the expression level of the at least one mRNA in the test sample to a level in a base sample for the same at least one mRNA, and
d) receiving a result comparing the expression levels of the at least one mRNA in the test sample measured in a) and the base sample measured in c), and
e) detecting or determining the prognosis of breast cancer based on altered expression of the least mRNA in the test sample as compared to the base sample.

2. The method of claim 1, further comprising a step of treating the subject based on the detection or prognosis of breast cancer.

3. The method of claim 2, wherein the treatment is one or more of surgery, radiation, chemotherapy, hormone therapy, targeted drug therapy and immunotherapy.

4. The method of claim 1, further comprising a step of distinguishing between stage 0, stage 1, stage 2, stage 3, stage 4 and stage 5 of breast cancer.

5. The method of claim 1, wherein the test sample is a blood sample or a saliva sample.

6. The method of claim 1, wherein the breast cancer is one or more of ductal carcinoma in situ, invasive lobular carcinoma in situ, tubular/cribriform carcinoma, mucinous (colloid) carcinoma, medullary carcinoma, papillary carcinoma and metaplastic carcinoma.

7. The method of claim 1, wherein the at least one mRNA is identified as one or more of SLC8A1, RP11-452L6.1, HINT3, PRORSD1P, PRDX6, GABRB2, EPS8, SH3RF1, GB_ACC AA814006, Hypothetical protein LOC284058, ZNF160, SLC25A45, DST, MTAP, GB_Acc AK024901, TFB1M, SPRED1, CAV1, MUC1, NAP1L3, MDH1B, TMEM125, PDE9A, TSHZ2, LAMB4 and PTGS2.

8. The method of claim 1, wherein the method is repeated to confirm the detection or prognosis of breast cancer.

9. The method of claim 1, wherein the at least one mRNA is comprised of 2, 3, 4, 5, 6, 7, 8, 9, 10, 11, 12, 13, 14, 15, 16, 17, 18, 19, 20, 21, 22, 23, 24, 25 or 26 mRNA biomarkers.

10. A method of detecting breast cancer or determining a prognosis of a test subject with breast cancer, comprising steps of:
a) measuring expression levels of two or more mRNAs in samples from subjects with breast cancer,
b) measuring expression levels of the two or more mRNAs in samples from healthy subjects,
c) using a computer to compare the expression levels of the two or more mRNAs in the samples from the subjects with breast cancer to the levels in the samples from the healthy subjects,
d) identifying mRNAs that have altered levels of expression in the plasma samples from the subjects with breast cancer,
e) creating a biomarker fingerprint from the mRNAs with altered levels of expression, and
f) diagnosing or determining the prognosis of breast cancer in a test subject by obtaining a score that sets forth results from comparing levels of mRNAs of the test subject to those in the biomarker fingerprint.

11. The method of claim 10, further comprising a step of treating the subject based on detection or prognosis of breast cancer.

12. The method of claim 11, wherein the treatment is one or more of surgery, radiation, chemotherapy, hormone therapy, targeted drug therapy and immunotherapy.

13. The method of claim 10, further comprising a step of distinguishing between stage 0, stage 1, stage 2, stage 3, stage 4 and stage 5 of breast cancer.

14. The method of claim 10, wherein the samples from subjects with breast cancer and samples from healthy subjects are blood samples or saliva samples.

15. The method of claim 10, wherein the breast cancer is one or more of ductal carcinoma in situ, invasive lobular carcinoma in situ, tubular/cribriform carcinoma, mucinous (colloid) carcinoma, medullary carcinoma, papillary carcinoma and metaplastic carcinoma.

16. The method of claim 10, wherein the two or more mRNAs are identified from genes in the group consisting of SLC8A1, RP11-452L6.1, HINT3, PRORSD1P, PRDX6, GABRB2, EPS8, SH3RF1, GB_ACC AA814006, Hypothetical protein LOC284058, ZNF160, SLC25A45, DST, MTAP, GB_Acc AK024901, TFB1M, SPRED1, CAV1, MUC1, NAP1L3, MDH1B, TMEM125, PDE9A, TSHZ2, LAMB4 and/or PTGS2.

17. The method of claim 10, wherein the two or more mRNAs are comprised of 2, 3, 4, 5, 6, 7, 8, 9, 10, 11, 12, 13, 14, 15, 16, 17, 18, 19, 20, 21, 22, 23, 24, 25 or 26 mRNA biomarkers.

18. The method of claim 10, wherein the method is repeated to create a second biomarker footprint to confirm the detection or prognosis of breast cancer.

19. A diagnostic kit for detecting breast cancer or determining a prognosis of a test subject with breast cancer,
wherein the kit comprises a plurality of nucleic acid molecules, each nucleic acid molecule encoding a mRNA sequence,
wherein the plurality of nucleic acid molecules identify variations in expression levels of one or more mRNAs in a sample from a test subject, and
wherein at least the plurality of nucleic acid molecules includes a plurality of base sample nucleic acid molecules, and
wherein the expression levels of one or more mRNAs represent a nucleic acid expression fingerprint that is indicative for the presence of and breast cancer, and
wherein the kit identifies one or more target cells exhibiting an mRNA expression from the sample from the test subject to detect breast cancer or determine a prognosis of a test subject with breast cancer.

20. The diagnostic kit of claim 19, wherein the breast cancer is one or more of ductal carcinoma in situ, invasive lobular carcinoma in situ, tubular/cribriform carcinoma, mucinous (colloid) carcinoma, medullary carcinoma, papillary carcinoma and metaplastic carcinoma.

21. The diagnostic kit of claim 19, wherein the one or more mRNAs are identified as genes from the group consisting of SLC8A1, RP11-452L6.1, HINT3, PRORSD1P, PRDX6, GABRB2, EPS8, SH3RF1, GB_ACC AA814006, Hypothetical protein LOC284058, ZNF160, SLC25A45, DST, MTAP, GB_Acc AK024901, TFB1M, SPRED1, CAV1, MUC1, NAP1L3, MDH1B, TMEM125, PDE9A, TSHZ2, LAMB4 and PTGS2.

22. The diagnostic kit of claim 19, wherein the test sample is a blood sample or a saliva sample.

23. The diagnostic kit of claim 19, wherein the breast cancer is one or more of ductal carcinoma in situ, invasive lobular carcinoma in situ, tubular/cribriform carcinoma, mucinous (colloid) carcinoma, medullary carcinoma, papillary carcinoma and metaplastic carcinoma.

24. The diagnostic kit of claim 19, wherein the one or more mRNAs are comprised of 2, 3, 4, 5, 6, 7, 8, 9, 10, 11, 12, 13, 14, 15, 16, 17, 18, 19, 20, 21, 22, 23, 24, 25 or 26 biomarkers.

25. A method for identifying one or more target cells exhibiting breast cancer in a patient, the method comprising steps of:
(a) collecting a patient sample from the patient;
(b) hybridizing one or more nucleic acid molecule biomarkers encoding a mRNA sequence to a portion of the patient sample;
(c) using a computer to quantify expression levels of one or more mRNAs;
(d) comparing the expression levels of the one or more mRNAs to expression levels in a control sample;
(e) compiling altered levels of expression of the one or more mRNAs;
(f) determining if the patient has a breast cancer and/or determining the prognosis of the breast cancer based on the altered levels of expression of the one or more mRNAs.

26. The method of claim 25, wherein the breast cancer is one or more of ductal carcinoma in situ, invasive lobular carcinoma in situ, tubular/cribriform carcinoma, mucinous (colloid) carcinoma, medullary carcinoma, papillary carcinoma and metaplastic carcinoma.

27. The method of claim 25, wherein the plurality of nucleic acid molecules are identified from the genes consisting of SLC8A1, RP11-452L6.1, HINT3, PRORSD1P, PRDX6, GABRB2, EPS8, SH3RF1, GB_ACC AA814006, Hypothetical protein LOC284058, ZNF160, SLC25A45, DST, MTAP, GB_Acc AK024901, TFB1M, SPRED1, CAV1, MUC1, NAP1L3, MDH1B, TMEM125, PDE9A, TSHZ2, LAMB4 and/or PTGS2.

28. The method of claim 25, wherein the one or more mRNAs are comprised of 2, 3, 4, 5, 6, 7, 8, 9, 10, 11, 12, 13, 14, 15, 16, 17, 18, 19, 20, 21, 22, 23, 24, 25 or 26 biomarkers.

29. The method of claim 25, further comprising a step of treating the subject based on the detection or prognosis of breast cancer.

30. The method of claim 29, wherein the treatment is one or more of surgery, radiation, chemotherapy, hormone therapy, targeted drug therapy and immunotherapy.

31. The method of claim 25, further comprising a step of distinguishing between stage 0, stage 1, stage 2, stage 3, stage 4 and stage 5 of breast cancer.

32. The method of claim 25, wherein the patient sample is a blood sample or a saliva sample.

33. The method of claim 25, wherein the method is repeated to confirm the detection or prognosis of breast cancer.

34. A method of diagnosing a breast cancer or determining a prognosis of a subject with a breast cancer, comprising the steps of:
a) measuring an expression level of at least one nucleic acid, protein or peptide in a test sample from plasma of the subject,
b) receiving the results of the measurement a) by a computer and
c) comparing the expression level of the at least one nucleic acid, protein or peptide in the test sample to a level in a base sample measure for the same at least one nucleic acid, protein or peptide, and
d) receiving a result comparing the expression levels of the at least one nucleic acid, protein or peptide in the test sample measured in a) and the base sample measured in c), and
e) diagnosing or determining the prognosis of breast cancer based on altered expression of the least one nucleic acid, protein or peptide in the test sample as compared to the base sample as determined in a computer.

35. The method of claim 34, wherein the at least one nucleic acid, protein or peptide is identified from the genes consisting of SLC8A1, RP11-452L6.1, HINT3, PRORSD1P, PRDX6, GABRB2, EPS8, SH3RF1, GB_ACC AA814006, Hypothetical protein LOC284058, ZNF160, SLC25A45, DST, MTAP, GB_Acc AK024901, TFB1M, SPRED1, CAV1, MUC1, NAP1L3, MDH1B, TMEM125, PDE9A, TSHZ2, LAMB4 and/or PTGS2.

36. The method of claim 34, wherein the breast cancer is one or more of ductal carcinoma in situ, invasive lobular carcinoma in situ, tubular/cribriform carcinoma, mucinous (colloid) carcinoma, medullary carcinoma, papillary carcinoma and metaplastic carcinoma.

37. The method of claim 34, further comprising a step of treating the subject based on detection or prognosis of breast cancer.

38. The method of claim 37, wherein the treatment is one or more of surgery, radiation, chemotherapy, hormone therapy, targeted drug therapy and immunotherapy.

39. The method of claim 34, further comprising a step of distinguishing between stage 0, stage 1, stage 2, stage 3, stage 4 and stage 5 of breast cancer.

40. The method of claim 34, wherein the test sample is a blood sample or a saliva sample.

41. A method of evaluating a probability a subject has breast cancer, diagnosing a breast cancer and/or monitoring breast cancer progression comprising steps of (a) measuring an amount of one or more mRNA biomarkers selected from the genes consisting of SLC8A1, RP11-452L6.1, HINT3, PRORSD1P, PRDX6, GABRB2, EPS8, SH3RF1, GB_ACC AA814006, Hypothetical protein LOC284058, ZNF160, SLC25A45, DST, MTAP, GB_Acc AK024901, TFB1M, SPRED1, CAV1, MUC1, NAP1L3, MDH1B, TMEM125, PDE9A, TSHZ2, LAMB4, PTGS2; (b) comparing the measured amount to a control and detecting an increase in the amount of the one or more mRNA biomarkers compared to a control; and (c) identifying the subject as having or having an increased probability of having the breast cancer when an increase in the one or more mRNA biomarkers compared to control is detected.

42. The method of claim 41, wherein the breast cancer is one or more of ductal carcinoma in situ, invasive lobular carcinoma in situ, tubular/cribriform carcinoma, mucinous (colloid) carcinoma, medullary carcinoma, papillary carcinoma and metaplastic carcinoma.

43. The method of claim 41, further comprising a step of treating the subject based on detection or prognosis of breast cancer.

44. A method of diagnosing a breast cancer or determining a prognosis of a subject with a breast cancer, comprising steps of:
a) measuring an expression level of at least one mRNA or miRNA in a test sample from the subject,
b) receiving of the expression level with a computer,
c) comparing the expression level in the test sample to a level in one or more base samples for the same at least one mRNA or miRNA,
d) receiving a result comparing the expression levels of the at least one mRNA or miRNA in the test sample measured in a) and the base sample measured in c), and
e) determining if the patient has breast cancer and/or determining the prognosis of the breast cancer based on altered expression of the at least one mRNA or miRNA in the test sample,
wherein the at least one mRNA or miRNA is identified from the genes consisting of SLC8A1, RP11-452L6.1, HINT3, PRORSD1P, PRDX6, GABRB2, EPS8, SH3RF1, GB_ACC AA814006, Hypothetical protein LOC284058, ZNF160, SLC25A45, DST, MTAP, GB_Acc AK024901, TFB1M, SPRED1, CAV1, MUC1, NAP1L3, MDH1B, TMEM125, PDE9A, TSHZ2, LAMB4 and PTGS2.

45. The method of claim 44, further comprising a step of treating the subject based on detection or prognosis of breast cancer.

46. The method of claim 44, wherein the breast cancer is one or more of ductal carcinoma in situ, invasive lobular carcinoma in situ, tubular/cribriform carcinoma, mucinous (colloid) carcinoma, medullary carcinoma, papillary carcinoma and metaplastic carcinoma.
